(19) United States
(12) Patent Application Publication
(10) Pub. No.: US 2009/0196909 A1
(43) Pub. Date: Aug. 6, 2009

(54) CARBON NANOTUBE CONTAINING MATERIAL FOR THE CAPTURE AND REMOVAL OF CONTAMINANTS FROM A SURFACE

(76) Inventors: Christopher H. Cooper, Windsor, VT (US); Whitmore B. Kelley, JR., Lebanon, NH (US); Vardhan Bajpai, Lebanon, NH (US); Daniel Iliescu, White River Junction, VT (US); Thomas H. Treutler, Sheffield, MA (US); Andrei Burnin, West Lebanon, NH (US); Hai-Feng Zhang, Winchester, MA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/256,302

(22) Filed: Oct. 22, 2008

Related U.S. Application Data
(60) Provisional application No. 60/981,924, filed on Oct. 23, 2007.

(51) Int. Cl. A61K 9/70; B08B 1/00; A61K 9/14; B05D 3/00; B32B 5/16; D03D 25/00; D04H 13/00; D04H 3/00; C11D 17/00; C08F 8/00; C08G 73/00; C07K 14/00; C07C 245/20; C07H 99/00; C07H 21/00; C07F 7/02; C07C 255/00; C07C 211/00; C07C 233/00; C07C 381/00; C07C 321/00; C07C 49/00; C07C 47/00; C07C 43/00; C07C 29/00; C01B 31/00

(52) U.S. Cl. 424/445; 134/6; 424/489; 427/352; 428/323; 442/181; 442/304; 442/417; 510/445; 525/50; 525/540; 530/402; 534/558; 536/1.11; 536/23.1; 556/400; 558/303; 564/1; 564/123; 568/38; 568/61; 568/303; 568/420; 568/579; 568/700; 977/742; 977/750; 977/752; 977/903

(57) ABSTRACT

There is disclosed an article and method of making an article for removing at least one contaminant from a solid surface. In one embodiment, the article comprises carbon nanotubes attached to a support media, such as a nonwoven mixture of PET and cotton. There is also disclosed a method of removing at least one contaminant from a solid surface, such as areas where microbial, particle, or static contamination is undesirable, including hospitals, clean rooms, kitchens, baths, or human hands.

This application claims the benefit of domestic priority to U.S. Provisional Patent Application Ser. No. 60/981,924 filed Oct. 23, 2007, which is herein incorporated by reference in its entirety.

Disclosed herein are carbon nanotube containing articles, such as wipes, for capturing and removing contaminants from a solid surface. Methods of making and methods of using such articles are also disclosed.

The promise of nano-science and nanotechnology as a whole, and nano-materials in particular, is that through the molecular scale control of material structures it will enhance the performance of traditional macro-scale materials, such as in the area of contaminant cleanup. Many of the current processes can be improved by using articles or wipes comprising nanomaterials, such as carbon nanotubes.

### SUMMARY OF INVENTION

It has been discovered that carbon nanotubes properly prepared and optionally attached to a support media can impart an enhanced capture affinity that enables the removal of a myriad of contaminants from surfaces. These contaminants include, but are not limited to fluids, particles, fibers, biological agents, radionuclides, static charge, or combinations thereof while achieving at least one additional benefit, such as improving the conductivity, absorbency, or tensile strength of the resulting article.

Thus, there is disclosed an article for removing at least one contaminant from a solid surface. In one embodiment, the article comprises a support media comprising carbon nanotubes in an amount sufficient to remove at least one contaminate from a solid surface, wherein a majority of said carbon nanotubes have at least one defect and/or have at least one functional group, molecule or cluster attached thereto.

The present disclosure also relates to methods of making such an article. In one embodiment, it comprises:

(a) contacting a support media with a suspension comprising one or more carbon nanotubes to form a carbon nanotube infused support media;

(b) heating said carbon nanotube infused support media to substantially dry said suspension;

(c) rinsing said support to remove loose carbon nanotubes; and

(d) drying said rinsed article.

In certain embodiments this article can be used as a wipe media with enhanced physical, chemical and electrical properties for cleaning surfaces that are contaminated with fluids, solvents, particles, fibers, biological agents, radionuclides, static charge, or combinations thereof.

This media, which in one embodiment is in the form of a wipe, is designed for the hygienic cleaning of contaminated surfaces, such as surfaces, products, equipment, tools, personnel, literature, and biological material in a clean room, industrial environment, clinical environment, household environment, office environment, military environment, public spaces, public transportation, vehicles, and academic environment. In certain embodiments, this media also has properties useful to electronic industries for removal or reduction of charge and/or charged particles from a surface for protection or manufacture of electronic components. In other embodiments, this media will be used in removing radioactive residues left on surfaces in laboratories or industries working with radioactive materials.

### BRIEF DESCRIPTION OF THE DRAWINGS

The accompanying figures are incorporated in, and constitute a part of this specification.

FIG. 1. Is a schematic structure of a Single-Walled Carbon Nanotube (SWCNT)

FIG. 2. Is a schematic of a distortion of the crystal lattice of the carbon atoms forming the tubular carbon nanotube structure.

FIG. 3. Is a schematic representation of the attachment of functional chemical groups, such as carboxyl groups, to the outer sidewalls of a carbon nanotube.

FIG. 4. Is a schematic showing the structural modification of the crystal lattice in a “doped” carbon nanotube.

FIG. 5. Is a schematic of a four point probe used for conductivity measurement.

FIG. 6. Is the structural formula of chelator molecule (EDTA) used in the radionuclide cleaning wipe.

FIG. 7. Is a representation of the confinement of the metal atom/cation (M) with a chelator molecule.

FIG. 8. Is a Scanning Electron Micrograph (SEM) showing the nanostructure of a MWC NT-polyester/cellulose wipe (DurX®670, by Berkshire).

FIG. 9. Is a Scanning Electron Micrograph (SEM) showing the attachment of carbon nanotubes to microfibers (LabX®170, by Berkshire).

FIG. 10. is a representation of the covalent bonding of a carbon nanotube to a LabX® media.

FIG. 11. Photograph of bio-results for the samples mentioned in Table 3.

### DETAILED DESCRIPTION OF INVENTION

There is provided in one aspect of the present disclosure an article containing carbon nanotubes for removing contaminants from a solid surface. “Contaminants” means at least one unwanted or undesired element, ion, molecule, particle or organism.

“Removing” (or any version thereof) is understood to mean capturing and retaining, destroying, or neutralizing contaminants using physical or chemical phenomenon chosen from but not limited to absorption, adsorption, entangling, and chemical or biological interaction or reaction.

“Chemical or biological interaction or reaction” is understood to mean an interaction with the contaminant through either chemical or biological processes that renders the contaminant incapable of causing harm. Examples of this are reduction, oxidation, chemical denaturing, and physical damage to microorganisms, bio-molecules, ingestion, and encasement.

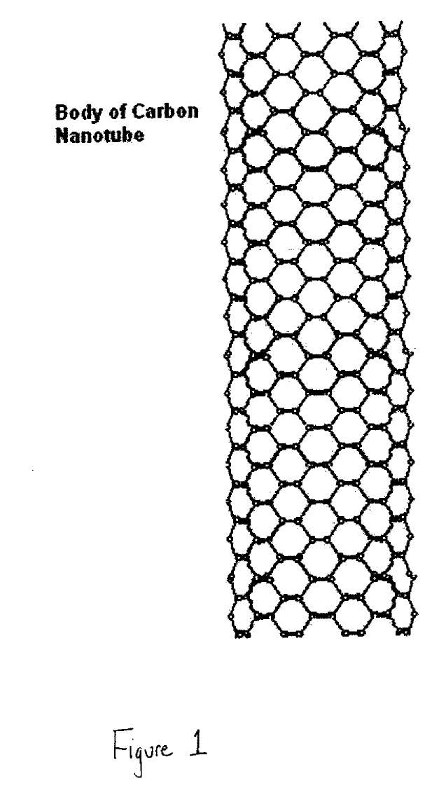

Carbon nanotubes are tubular structures composed of one or many seamless, concentric, rolled sheets of graphene that may be open on both ends or terminated on one or both ends by hemispherical fullerene cap(s). Carbon nanotubes composed of one graphene sheet, as depicted in FIG. 1, are termed “single walled carbon nanotubes” (SWCNTs) and those of many concentric sheets are termed “multiwalled carbon nanotubes” (MWCNTs). Single-walled carbon nanotubes are generally around 1-2 nm in diameter, similar to human DNA (˜2 nm), while multi-walled carbon nanotubes can have diameters of tens of nanometers. Both types of carbon nanotubes can theoretically be of any length, but usually range from 5 nm to a few millimeters and even centimeters in length.

One aspect of the present disclosure is related to the use of carbon nanotubes that have a scrolled tubular or non-tubular nano-structure of carbon rings. These carbon nanotubes may be single-walled, multi-walled or combinations thereof, and may take a variety of morphologies. For example, the carbon nanotubes used in the present disclosure may have a morphology chosen from horns, spirals, multi-stranded helices, springs, dendrites, trees, spider nanotube structures, nanotube Y-junctions, bamboo morphology and the like. Some of the above described shapes are more particularly defined in M. S. Dresselhaus, G. Dresselhaus, and P. Avouris, eds. Carbon Nanotubes Synthesis, Structure, Properties, and Applications, Topics in Applied Physics. 80. 2000, Springer-Verlag; and “A Chemical Route to Carbon Nanoscrolls, Lisa M. Viculis, Julia J. Mack, and Richard B. Kaner; Science, 28 Feb. 2003; 299, both of which are herein incorporated by reference.

Particles removed from the surface by the inventive article may be of a size ranging from sub-nanometers to a few millimeters. “Particle size” being defined by a number distribution, e.g., by the number of particles having a particular size. The method is typically measured by microscopic techniques, such as by a calibrated optical microscope, by calibrated polystyrene beads, by calibrated scanning probe microscope scanning electron microscope, or optical near field microscope. Methods of measuring particles of the sizes described herein are taught in Walter C. McCrone's et al., The Particle Atlas, (An encyclopedia of techniques for small particle identification), Vol. I, Principles and Techniques, Ed. 2 (Ann Arbor Science Pub.), which is herein incorporated by reference.

Non-limiting examples of contaminants that may be removed from a surface using the disclosed article includes, but is not limited to: fluids, particles, fibers, biological agents, radionuclides, static charge, or combinations thereof, such as viruses, bacteria, fungi, molds, organic and inorganic chemical contaminants (both natural and synthetic) or ions. In one embodiment, the fluids are comprised of water, hydrocarbons, acid, fluids, radioactive wastes, foodstuffs, bases, solvents or combinations thereof. In another embodiment, the radionuclides comprise at least one atom or ion chosen from the elements: strontium, iodine, cesium, beryllium, lithium, sodium, barium, polonium, radium, thorium, hydrogen, uranium, plutonium, cobalt, and radon. In yet another embodiment, the biological agents comprise molecules chosen from DNA, RNA, and natural organic molecules bacteria, viruses, spores, mold, parasites, pollens, fungi, prion and combinations thereof. It is understood that any known bacteria may be removed, including anthrax, coliforms typhus, e-coli, staph, pneumonia, salmonella, or cholera. Similarly, any form of virus may be removed, including smallpox, hepatitis, or HIV and their variants.

Further, the article achieves this contaminant removal while achieving at least one additional benefit, at least partly due to the presence of carbon nanotubes, such as improving the conductivity of the article, the absorbency of the article or increasing the tensile strength of the resulting article.

In one embodiment, the disclosed article is composed of one or more layers in which the composition varies between and/or within layers such that the concentration of the carbon nanotubes may vary from 0.01% to 99% by weight and may be different in each layer.

In one embodiment the disclosed article is impregnated with carbon nanotubes on the surface or throughout the depth of the support media so that the microbial capture properties of carbon nanotubes are utilized to enhance the cleaning properties of the support media.

In one embodiment of the disclosed article, a majority of the carbon nanotubes are distorted by crystalline defects such that they exhibit a greater contaminant removal affinity than non-distorted carbon nanotubes. “Crystalline defects” refers to sites in the tube walls of carbon nanotubes where there is a lattice distortion in at least one carbon ring.

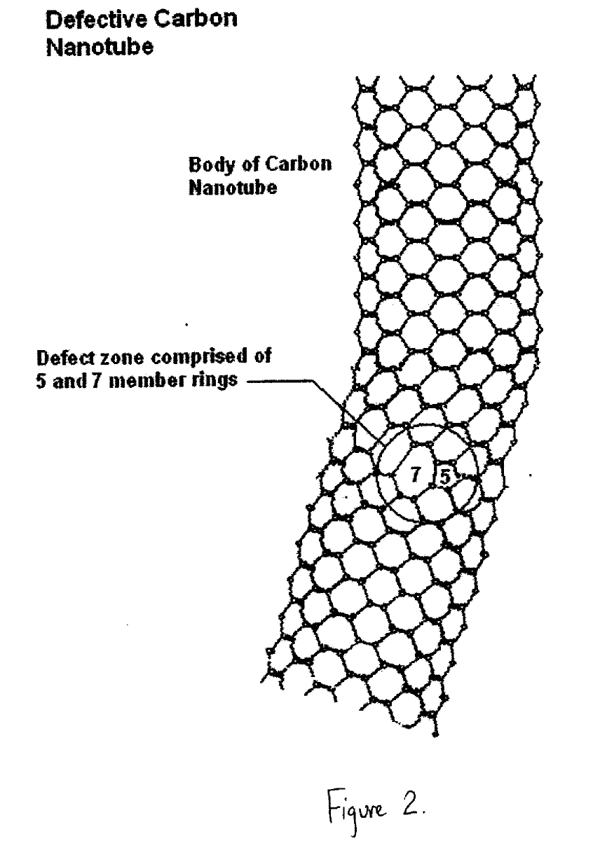

A “lattice distortion” means any distortion of the crystal lattice of carbon nanotube atoms forming the tubular sheet structure. As exemplified in FIG. 2, a lattice distortion may include any displacements of atoms because of inelastic deformation, or presence of 5 and/or 7 member carbon rings, or a chemical interaction followed by change in hybridization of carbon atom bonds Such defects or distortions may lead to a natural bend in the carbon nanotube.

The phrase “exhibit a greater contaminant removal affinity” means that by virtue of the changes realized in the structural integrity, its porosity, its pore size distribution, its electrical conductance, its resistance to fluid flow, geometric constraints, capture capacity or any combination thereof, due to use of carbon nanotube in the inventive media leads to an enhancement of contaminant removal. For example, greater contaminant removal affinity could be due to improved and more efficient adsorption or absorption properties of the individual carbon nanotubes. Further, the more defects there are in the carbon nanotubes, the more sites exist for attaching chemical functional groups.

In one embodiment, increasing the number of functional groups present on the carbon nanotubes improves the removal affinity of the resulting article. The present disclosure also relates to a method of cleaning surfaces by contacting the contaminated surface with the article described herein. In one embodiment, the method of cleaning the surface comprises contacting the surface with a “inventive article”, wherein the carbon nanotubes are present in the same in an amount sufficient to reduce the concentration of at least one contaminant on the contacted surface to a level below that of the untreated surface after being treated with the inventive article; such as reducing the concentration by at least 50%, such as at least 75%, or even up to 100% removal of the contaminant initially present on the surface.

Applications for the articles described herein include hygienic cleaning of contaminated areas, such as surfaces, products, equipment, tools, personnel, literature, and biological material in a clean room, industrial environment, clinical environment, household environment, office environment, military environment, public spaces, public transportation, vehicles, and academic environment. In certain embodiments, this media also has properties useful to electronic industries for removal or reduction of charge and/or charged particles from a surface for protection or manufacture of electronic components. In other embodiments, this media will be used in removing radioactive residues left on surfaces in laboratories or industries working with radioactive materials.

In certain embodiments, the article described herein may be used in the following non-limiting locations: home (e.g. domestic surface disinfection, such as surfaces of bathrooms, kitchens, phones and door knobs), recreational (e.g. surface treatment of children's toys, sporting goods, camping applications), industrial (e.g. antistatic wipes, solvent reclamation, toxic chemical clean-up), governmental (e.g. waste remediation, material decontamination), and medical (e.g. operating rooms disinfection, wound and surgical preparation).

In various embodiments, the disclosed article may take the form of a disposable wipe, reusable cloth, article of clothing, swab, mop, brush, pad, or wound dressing. Within these forms, the inventive article may be made anti-microbial, anti-viral, anti-static, or combinations thereof.

In another embodiment, the inventive article may be pre-saturated with a liquid to further enhance the removal of a contaminant from a surface. Methods of using such an article are also disclosed. Alternatively, methods of wetting the article, or the surface to be cleaned prior to contacting it with the inventive articles are also disclosed. For example, in one embodiment, there is disclosed a method in which a liquid is applied to at least one of the article or the solid surface prior to contacting.

Non-limiting examples of the liquid that may be used include aqueous or non-aqueous solutions of alcohols, surfactants, detergents, and disinfectants.

### Treatment of Carbon Nanotubes

In the present disclosure, the carbon nanotubes may also undergo chemical and/or physical treatments to alter their chemical and/or physical behavior. For example, in one embodiment, the carbon nanotubes are chemically treated with an oxidizer, chosen from but not limited to a gas containing oxygen, nitric acid, sulfuric acid, hydrogen peroxide, potassium permanganate, and combinations thereof. Carbon nanotubes which have been treated with an oxidizer can provide unique properties, either in terms of chemical capture affinity, dispersion of nanotubes in the deposition fluid, or from a functionalization perspective (e.g., having the ability to be particularly functionalized). These treatments are typically done to enable the resulting article to exhibit greater contaminant removal affinities, in the sense defined above.

The treatments described herein enable at least one molecule or cluster comprising, for example, an organic compound chosen from proteins, carbohydrates, polymers, aromatic or aliphatic alcohols, nucleic acid, or combinations thereof, to be attached to the carbon nanotubes.

As used herein, “chemical or physical treatment” means treating with an acid, solvent an oxidizer, plasma treatment or radiation for a time sufficient to 1) remove unwanted constituents, such as amorphous carbon, oxides or trace amounts of by-products resulting from the carbon nanotube fabrication process; 2) to create increased defect density on the surface of the carbon nanotube; or 3) to attach specific functional groups that have a desired zeta potential (as defined in Johnson, P. R., Fundamentals of Fluid Filtration, 2^{nd} Edition, 1998, Tall Oaks Publishing Inc., which is incorporated herein by reference). These chemical treatments will act to change the surface chemistry of the carbon nanotubes sufficiently to increase the removal affinity of the inventive article for a specific set of target contaminants from a surface.

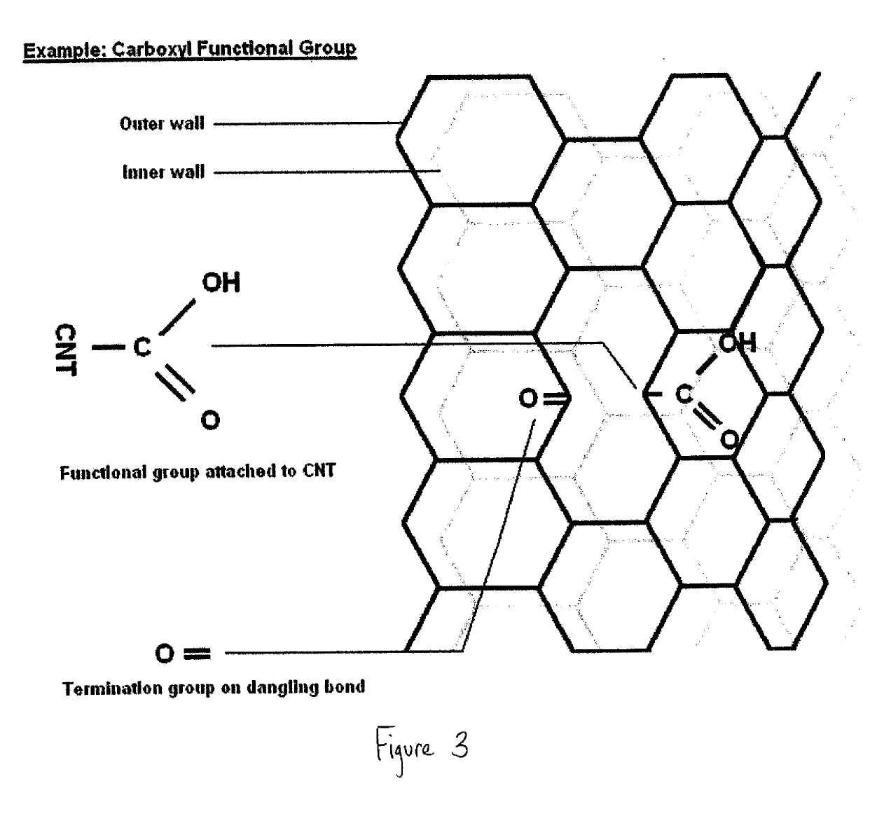

As used herein, “functionalized” (or any version thereof) refers to a carbon nanotube having an atom or group of atoms attached to the surface that may alter the properties of the nanotube, such as zeta potential. Functionalization is generally performed by modifying the surface of carbon nanotubes using chemical techniques, including wet chemistry or vapor, gas or plasma chemistry, and microwave assisted chemical techniques, and utilizing surface chemistry to bond materials to the surface of the carbon nanotubes. These methods are used to “activate” the carbon nanotube, which is defined as breaking at least one C—C or C-heteroatom bond, thereby providing a surface for attaching a molecule or cluster thereto. As shown in FIG. 3, functionalized carbon nanotubes comprise chemical groups, such as carboxyl groups, attached to the surface, such as the outer sidewalls, of the carbon nanotube. Further, the nanotube functionalization can occur through a multi-step procedure where functional groups are sequentially added to the nanotube to arrive at a specific, desired functionalized nanotube.

The functionalized carbon nanotubes can comprise a non-uniform composition and/or density of functional groups including the type or species of functional groups across the surface of the carbon nanotubes. Similarly, the functionalized carbon nanotubes can comprise a substantially uniform gradient of functional groups across the surface of the carbon nanotubes. For example, there may exist, either down the length of one nanotube or within a collection of nanotubes, many different functional group types (i.e. hydroxyl, carboxyl, amide, amine, poly-amine and/or other chemical functional groups) and/or functionalization densities.

In another embodiment, the carbon nanotubes contain atoms, ions, molecules or clusters attached thereto or located therein in an amount sufficient to assist in the removal and/or modification of contaminants from the surface.

Further, other components of the article such as fibers and/or nanoparticles, may also be functionalized with chemical groups, decorations or coatings or combinations thereof to change their zeta potential and/or cross-linking abilities and thereby improve the contaminant removal performance of the article.

A non-limiting example of performing a specific functionalization is one where the carbon nanotubes are refluxed in a mixture of acids which allows the zeta potential of carbon nanotubes to be modified thereby improving their ability to remove and/or retain contaminants. While not being bound by any theory, it is believed that such a process increases the number of defects on the surface of the nanotube, attaches carboxyl functional groups to the carbon nanotube's surface at the defect sites thereby changing the zeta potential of the nanotubes due to the negative charge character of carboxyl functional groups in water.

In another embodiment, carbon nanotubes can also be used for high surface area molecular scaffolding either for functional groups comprised of organic and/or inorganic receptors or to provide structure and support for natural or bioengineered cells [including bacteria, nanobacteria and extremophilic bacteria]. Examples of nanobacteria, including images of nanobacteria in carbonate sediments and rocks can be found in the following references, which are herein incorporated by reference. R. L. Folk, J. Sediment. Petrol. 63:990-999 (1993), R. H. Sillitoe, R. L. Folk and N. Saric, Science 272:1153-1155 (1996).

The addition of functional groups containing specific organic and/or inorganic receptors will selectively target the removal of specific contaminants from the surface. The natural or bioengineered cells supported by the nanotubes will consume, metabolize, neutralize, and/or bio-mineralize specific biologically-active contaminants.

In another aspect of this invention, the carbon nanotubes, the carbon nanotube material, or any sub-assembly thereof may be treated with electromagnetic or particle beam radiation. In this embodiment, the radiation impinges upon the carbon nanotube in an amount sufficient to 1) break at least one carbon-carbon or carbon-heteroatom bond; 2) perform cross-linking between nanotubes, nanotube and other nanomedia constituent, or nanotubes and the substrate; 3) perform particle implantation, 4) induce chemical treatment of the carbon nanotubes, or any combination thereof. Irradiation can lead to a differential dosage of the nanotubes (for example due to differential penetration of the radiation) which causes non-uniform defect structure within the nanomedia structure. This may be used to provide a variation of properties, via a variation of the density of functional groups and/or particles attached to the carbon nanotubes.

In addition, carbon nanotubes, according to the present disclosure, may be modified by coating or decorating with a material and/or one or many particles to assist in the removal of contaminants from the surface or increase other performance characteristics such as mechanical strength, bulk conductivity, or nano-mechanical characteristics. Coated or decorated carbon nanotubes are covered with a layer of material and/or one or many particles which, unlike a functional group, is not necessarily chemically bonded to the nanotube, and which covers a surface area of the nanotube sufficient to improve the contaminant removal performance of the article. As used herein “decorated” refers to a partially coated carbon nanotube. A “cluster” means at least two atoms or molecules attached by any chemical or physical bonding.

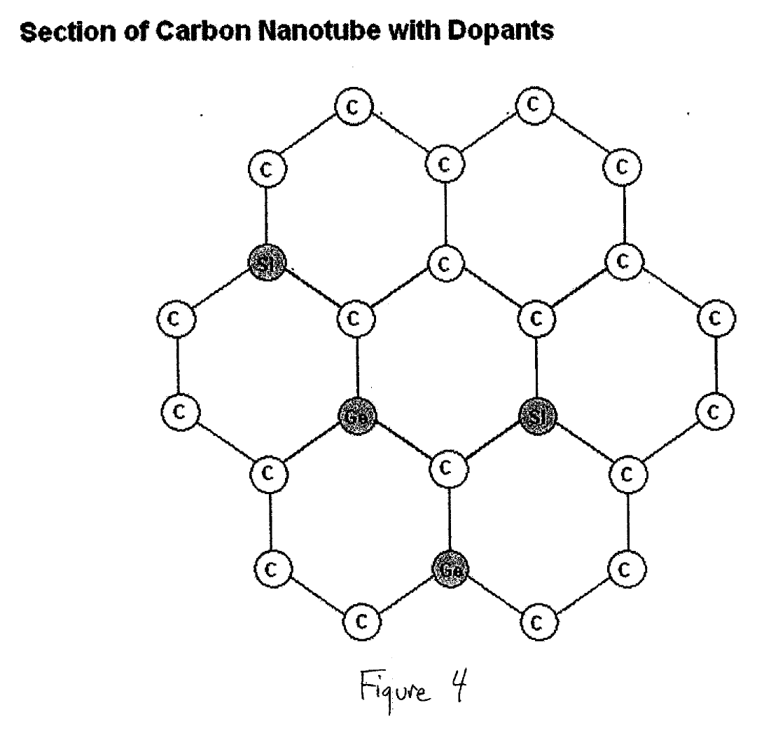

Carbon nanotubes used in the article described herein may also be doped with constituents to assist in the removal of contaminants from fluids. As used herein, a “doped” carbon nanotube refers to the presence of ions or atoms, other than carbon, into the crystal structure of the rolled sheets of hexagonal carbon. As exemplified in FIG. 4, doped carbon nanotubes means at least one carbon in the hexagonal ring is replaced with a non-carbon atom.

### Support Media

The support media described herein may include a fibrous material, such as paper or a textile comprised of a woven construction, a knit construction, a nonwoven construction, or a combination thereof.

In one embodiment, the textile may be comprised of multi-component or bi-component fibers or yarns which may be splittable along their length by chemical or mechanical action.

In another embodiment, the textile is comprised of microdenier fibers. The textile may also be comprised of synthetic fibers, natural fibers, man-made fibers using natural constituent, or blends thereof.

In another embodiment, the natural fibers are comprised of wool, cotton, silk, ramie, jute, flax, abaca, wood pulp, or blends thereof.

The man-made fibers described herein may comprise natural constituents, such as regenerated cellulose, lyocell or blends thereof.

The polymeric materials that may make up the synthetic fibers include single or multi-component polymers chosen from polyester, acrylic, polyamide, polyolefin, polyaramid, polyurethane or blends thereof. Other materials may include nylon, acrylic, methacrylic, epoxy, silicone rubbers, polypropylene, polyethylene, polyurethane, polystyrene, aramids, polycarbonates, polychloroprene, polybutylene terephthalate, poly-paraphylene terephtalamide, poly (p-phenylene terephtalamide), and polyester ester ketone, polyesters, polytetrafluoroethylene, polyvinylchloride, polyvinyl acetate, viton fluoroelastomer, polymethyl methacrylate, polyacrylonitrile, and combinations thereof.

In one embodiment, the carbon nanotube containing “inventive article” contains synthetic fibers. Non-limiting examples of such synthetic fibers include polyolefins, such as polyethylene, polypropylene, and polybutylene, halogenated polymers, such as polyvinyl chloride, polyesters, such as polyethylene terephthalate (PET), polyester/polyethers, polyamides, such as nylon 6 and nylon 6,6, polyurethanes, as well as homopolymers, copolymers, or terpolymers in any combination of such monomers, and the like. The combination of polyethylene terephthalate (PET) and cellulose fibers (sold under the tradename DURX 670® by Berkshire) are particularly noted as useful support media.

As stated, the foregoing materials may be fabricated in any known form, including but not limited to knitted, woven, non-woven, film, foam, paper, and/or combinations thereof.

### Mechanisms of Action

Without wishing to be bound by any theory, it is believed that the “article” described herein forms a unique nanoscopic interaction zone that uses chemical and/or physical forces to attract and capture microbes, pathogens or chemical contaminants from the surface. It is possible that the surface contact forces disrupt the cell membranes or cause internal cellular damage, thus disabling and/or destroying the microorganisms or their ability to reproduce. Since the osmotic pressure within a typical microbial cell is higher than that of the surrounding fluid, assuming non-physiological conditions, even slight damage to the cell wall can cause total rupture as the contents of the cell flow from high to low pressure.

Further, without being bound by theory it is believed that carbon nanotubes destroy the ability of bacteria and viruses to reproduce or infect host cells rendering it incapable of causing infection. In this way, surfaces can be effectively sterilized with respect to microorganisms.

Further, the ability to chemically functionalize the carbon nanotubes contained in the inventive article with specific chemical groups allows for introducing active contaminant capture through the use of chemical processes. One non-limiting example of chemical capture is the action of chelators that contain specific contaminant traps that engulf chemical agents and immobilize the contaminant.

In one embodiment of the present invention, there is disclosed a wipe for cleaning radioactive materials. Such wipes would fill the need for the decontamination of surfaces from radioactive materials in industries ranging from nuclear power plants to high-tech research laboratories to hospitals using contrast agents in diagnostic tools.

An example of using a functionalized carbon nanotube containing article as described herein for the removal of radioactive contamination from surfaces includes: 1) application of a surfactant solution in order to separate contamination from the surface and 2) absorption of the contaminated liquid phase with either porous or gel-like hydrophilic media which is disposed upon saturation.

Since carbon nanotubes have very large affinity to hydrophobic tails of surfactant molecules, they can be effectively used for capturing such molecules after they bond to a contaminant. This should provide specific removal of radioactive contaminants bound to surfactant moieties from surfaces without absorbing excessive amounts of solvent, such as water.

A suitable grade of carbon nanotubes necessary to create mats used in this embodiment would be those long enough to be able to lock in a buckypaper like structure. Later this material can be enhanced by addition of shorter carbon nanotubes cross-linked to the longer ones. In another embodiment, the multi-walled carbon nanotubes can be functionalized with very bulky silica gel species or super absorbent polymers (SAP), which would provide absorption of the whole amount of the cleaning liquid from the surface.

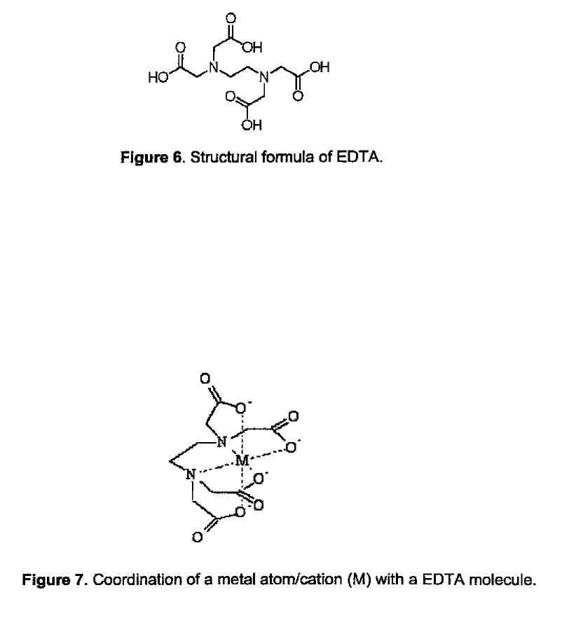

In order to provide specific adsorption of heavy metal contaminants and their radioactive isotopes, chelation chemistry is employed. In one embodiment, carbon nanotubes can be functionalized with derivatives of Ethylene diamine tetraacetic acid, EDTA, (FIG. 6). Such molecules, being polydentate ligands, provide multiple bonds to a metal atom via several coordination sites and should enable very effective capture of the corresponding impurities if these ligands are immobilized covalently on the surface of carbon nanotubes. A schematic of this is provided in FIG. 7, nanotube is not shown for simplicity.

The present disclosure is further illustrated by the following non-limiting examples, which are intended to be purely exemplary of the disclosure.

### EXAMPLES

### Example 1

### Inventive Surface Wipe

This example describes the fabrication of a wipe made according to one aspect of the present invention, particularly, one comprised of carbon nanotubes (CNTs) integrated into a non-woven cloth composed of a blend of polyethylene terephthalate (PET) polymer fibers and cellulose fibers. Such a non-woven material is commercially available and is sold under the tradename DURX 670® by the Berkshire Corporation. As described below, due to the unique properties of high surface area and electrical conductivity, it has been shown that the addition of carbon nanotubes to the non-woven cloth enhances its water retention and anti-static performance.

Overview of the Inventive Surface Wipe

Both short MWCNTs (˜1-50 μm in length) and extra long MWCNTs (˜3-5 mm in length) were chemically functionalized and then dispersed in water containing a negatively charged ionic surfactant using ultrasonication and high pressure (10,000-20,000 psi) microfluidization techniques. The negative surfactant was specifically chosen to be easy to rinse out of the final article.

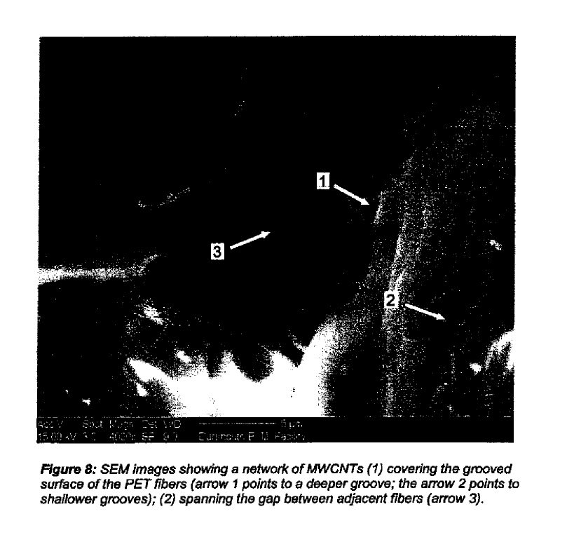

The role of the extra long MWCNTs was to bridge the gap between neighboring PET and cellulose fibers in the cloth (FIG. 8) and increase the electrical conductivity of the cloth. The role of the short MWCNTs was to interleave within the non-woven fiber matrix and bond with surface roughness elements, such as grooves and crevices, of the PET and cellulose fibers in the non-woven cloth.

Because of their larger size, the XL bundles did not readily penetrate deep into the crevices and grooves on the surface of the polymer fibers, even in the presence of surfactant. Thus, when used the XL carbon nanotubes were used alone, the cloth had a spotty distribution of active material and inconsistent electrical properties. The shorter MWCNTs on the other hand penetrated deep into the surface crevices linking whenever possible the XL bundles with the polymer fiber. However, the shorter MWCNTs alone did not have the length to easily achieve long range electrical conductivity.

To achieve an integrated structure with more uniform properties a mixture of extra long and short CNTs was used. The result of this approach was a uniformly gray cloth with relatively similar electrical properties across its surface. In addition, because of the deeper penetration of the short MWCNTs into and bonding with the non-woven media, this approach reduced shedding of carbon material and significantly increased in-plane electrical conductivity, when compared to the use of shorter CNT's alone.

### Manufacturing Procedure:

Preparation of the MWCNT Suspension

Prior to using, 1 g of untreated short MWCNTs were dispersed in 1000 ml of reversed osmosis (RO) water and mechanically functionalized using a high (20 kpsi) differential pressure microfluidizing device with a Z type processing chamber possessing a 100 μm orifice.

A 200 mg batch of XL MWCNTs was chemically functionalized by washing it in 70% nitric acid for 1 hour at 80° C. in a glass flask immersed in a Branson water bath sonicator. This process was known to attach carboxyl groups to the surface of MWCNTs. These functionalized XL MWCNTs were then rinsed with RO water until a pH of at least 5.5 was reached. The rinsed XL MWCNTs were then suspended in 1000 ml of RO water containing 1% by weight negative ionic surfactant. This mixture was sonicated for 15 minutes on high power before being passed through the high differential pressure microfluidizing device.

A 2000 ml suspension was produced by combining the 1000 ml suspension of the 1 g/L short MWCNTs and the 1000 ml suspension of the 0.2 g/L XL MWCNTs. The resulting 2000 ml mixture was probe sonicated on high power for 15 minutes using a Branson 900 BCA type sonicator. This mixed MWCNT suspension is referred to as the MWCNT-ink.

Pre-Preparation of the Base Cloth Media

5.5″×5.5″ squares of DURX 670® in as-received condition were soaked in water and bath sonicated for 15 minutes. This step 1) cleaned the surface of the cloth media; 2) loosened up the structure of the cloth and separates fibers which otherwise may have been joined together in tight bundles and; 3) helped reveal local topography (groove, crevices, etc) on the surface of the fibers. All of these effects helped increase the effective surface onto which the MWCNTs attached in the resulting article.

### Production of the CNT-Infused Article

Individual 5.5″×5.5″ pieces of the pre-prepared DURX 670® non-woven base media were infused with MWCNTs by tumbling them for 15 minutes in 2 liters of

MWCNT-containing ink using a magnetic stirrer.

The MWCNT-infused article was then removed from the MWCNT-containing ink and laid flat on Aluminum foil. The Aluminum foil with the MWCNT-infused article was then placed in an oven and heated at 110-115° C. for 30 minutes.

Preliminary tests with the as-received DURX 670® showed that heating above 100° C. caused the fabric to shrink macroscopically by approximately 5% primarily along the directional texture of the material. It was assumed that as the polymer fibers shrink the space between fibers and the grooves and crevices on their surface would also significantly reduce their size leading to a better retention of the CNTs inside the fiber structure of the cloth.

After heating and drying, the CNT-infused cloth was “tumble-washed” again in running clean water for 30 minutes, to rinse any unincorporated MWCNTs from the nanomedia, and again placed on Aluminum foil and dried in an oven at 60-90° C. for 30 minutes.

### Assessment Procedures:

Water Retention

The 5.5″×5.5″ squares of DURX® 670 in as-received condition were compared to processed pieces of the CNT-infused DURX® 670. The processed material was originally 5.5″×5.5″ in size but shrinking occurs during the heat treatment reducing somewhat the geometric area of these pieces of material but maintaining their mass.

To remove adsorbed moisture from the different media samples, “dry” samples of both processed and as-received cloths were placed in a vacuum oven and heated to 90° C. for 15 minutes. After that each piece of material was weighed individually and then fully immersed in water for ˜30 seconds. Each piece of cloth was removed from the water by pulling it with tweezers from two adjacent corners. During this process the cloth was kept in contact with the rim of the beaker in order to remove the excess water.

After holding the material suspended in air for 30 seconds the total weight was measured. This dipping and weighing procedure was performed 5-10 times by two persons for each cloth sample. The weight after dipping and the water content was computed by subtracting out the initial dry weight of each sample and averaged.

Electrical Resistance Measurements

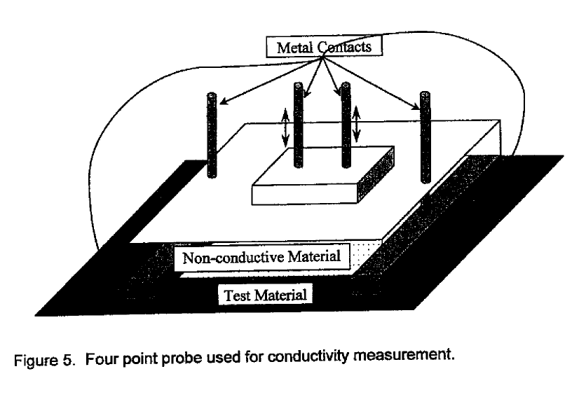

The sheet electrical resistance was measured using a 2″×2″ four point probe (FIG. 5) which pressed against the material using the same weight in all cases.

Antimicrobial Testing

Both the MWCNT treated DURX® 670 cloths and untreated DURX 670 cloths were placed in sterile 1 L bottles and immersed in 70% ethanol for about 5 minutes. The liquid was then drained out and the bottles placed in a clean oven at 50° C. for about an hour. At the end of this period both the as-received cloth and the one containing CNTs were completely dry.

An E. coli stock of about 10⁸ CFU/ml was reduced to 10⁶ CFU/ml by a 1:100 dilution. Using sterile swabs the liquid containing bacteria was smeared onto two sterile glass plates which were then wiped dry using the two 1″×1″ samples of cloth (with and without MWCNTs) held with sterile tweezers. The two pieces of cloth were placed in tubes containing 10 ml of Trypic Soy Broth (TSB) growth media. The glass plates were also checked for traces of bacteria by wiping their surface with swabs dipped in TSB broth. Similarly, the swabs were placed in 10 ml of TSB broth. All specimens including negative controls were incubated at 37° C. overnight.

A summary of the previously described tests is provided in Table 1.

TABLE 1

| | Base DURX ® 670 | Light Recipe | Medium Recipe | Dual CNT Recipe |
|---|---|---|---|---|
| Active material | PET-cotton | Short MWCNTs | Short MWCNTs | Short and XL |
| | | | | MWCNTs |
| Appearance | White | Light gray | Dark gray | Very light gray |
| BET surface area | 1.3-1.4 m²/g | ~1.8 | ~2.5 | ~1.8 |
| [m²/g] | | | | |
| Water retention | — | Base + ~15% | Base + ~20% | Base + ~8% |
| Antistatic | None | Poor | Good | Excellent |
| Performance | (R~∞) | (R > 90 MΩ/sq) | (R = 40-400 kΩ/sq) | (R = 2-20 kΩ/sq) |
| (Sheet resistance) | | | | |
| Bacterial | Inactivation on | Not tested | Inactivation on | No inactivation |
| Inactivation | glass substrate, | | glass substrate | |
| | not in cloth | | and in cloth | |

The above described results show that the negative controls taken from both the as-received DURX® 670 cloth and the CNT-containing cloth showed no sign of bacteria. The TSB growth medium was clear. The untreated polymer-cotton cloth used to wipe the bacteria-containing liquid off the glass plate surface did not inhibit further bacterial growth. The TSB growth medium was cloudy. In contrast, the CNT-containing cloth did inhibit bacterial growth. It is unknown if the bacteria was killed or just inactivated, however, the TSB growth medium was clear. In addition, both glass plates tested negative for bacteria. The TSB growth medium was clear.

### Example 2

### Covalently bonded Antimicrobial, Antistatic, Adsorptive Article

This example describes the fabrication of an antimicrobial, antistatic, adsorptive wipe made according to one aspect of the present invention, particularly, one comprised of carbon nanotubes (CNTs) integrated into a non-woven LabX®170 cloth. A media comprised of multi-walled carbon nanotubes (MWCNTs) with an added monomer functional group is integrated into LabX® 170 media for the purpose of enhancing the anti-static discharge and anti-microbial properties of the LabX® 170 wipe media.

### Manufacturing Procedure:

Functionalization of CNTs

5 mg of raw, short MWCNTs were oxidized in 70% nitric acid for 2 hrs at 80° C. These carboxylated MWCNTs were serially washed to remove residual acid with RO water until a pH of at least 5.5 was reached in the wash water. The washed MWCNTs were then re-suspended in 483 ml of RO water.

15 ml of HCL and 2 ml of Glycol were added sequentially to bring the volume of the suspension to 500 ml. Next, the suspension was sonicated for 1 hr with a BRANSON 900 BCA sonicator at 75% efficiency (8.45 KWH). 2.5 grams of hexyl decyl tri-ammonium bromide (HDTAB) surfactant was added and the suspension was sonicated for an additional 10 min to obtain a well-mixed 500 ml glycol functionalized MWCNT suspension.

Sample Preparation through Self-Assembly

2″×2″ non-woven fabric (LabX® wipe media) pieces were cut and soaked in 2% HCL solution at 70° C. for 2 hrs. These acid treated fabric pieces were then suspended into a 500 ml suspension of glycol-modified carbon nanotubes. Sample cloth pieces were removed from the suspensions at different times, rinsed several times with RO water and then dried at 100° C. for 4 hrs.

### Characterization:

Scanning Electron Microscopy

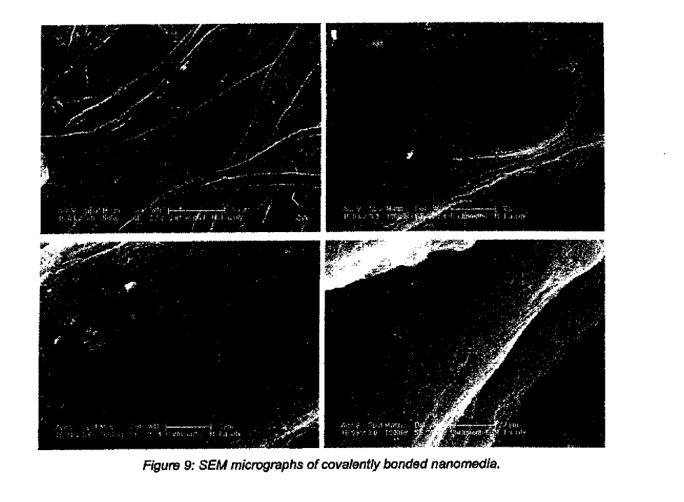

SEM Images were taken for the self-assembled MWCNTs on LabX wipe media. It was found that LabX wipe media is mainly made-up of fibers. SEM images show that polymer fibers in the labx 170 media are well coated with the carbon nanotubes which appear to be well integrated into/attached to the surface (FIG. 9).

### Thermogravimetric Analysis

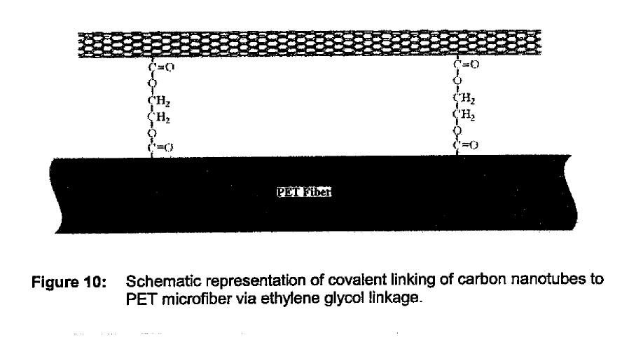

Results obtained from TGA analysis give the degree of chemical functionalization achieved on the surface of the carbon nanotubes. As shown in Table 2, it was found that during the oxidation step, which primarily adds carboxyl groups onto the surface of carbon nanotubes, a 0.8% by weight increase in the functionalization was obtained. A further 0.3% by weight increase was observed after the reaction with ethylene glycol molecule. Thus 0.3% increase in the weight is attributed to the ethylene glycol linkages as shown in the FIG. 10.

TABLE 2
Composition of the acid washed- glycol
functionalized MWCNT samples.

| | | Carboxyl | Glycol | Other |
|---|---|---|---|---|
| Sample | MWCNT | groups | groups | Impurities |
| LabX-glycol-MWCNT | 96.8% | 0.8% | 0.3% | 2.4% |

### Electrical Resistance Measurements

Resistance of the LabX® wipe media, measured as described in Example 1 above, changed measurably after the incorporation of carbon nanotubes into the non-woven media. The untreated LabX® samples were found to be non-conducting (Resistance ˜∞) whereas the MWCNT treated LabX® wipe media was found to be relatively conductive (resistance ˜30 kΩ/square).

Biotesting Results

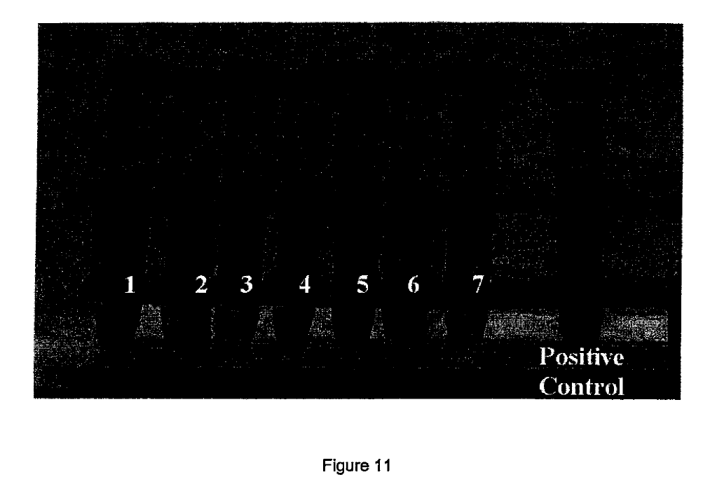

The sterility controls showed no growth at any time between 24 hours and 7 days from inoculation. The Table 2 lists the set of samples tested together with the positive growth control showed growth after overnight incubation. The cloudy appearance of rightmost tube is noted depicting the growth of the bacteria. Also, note clarity of solution the 7 solutions to the left in FIG. 2 depicting NO bacterial growth in the solution. None of the tubes containing the 1″×1″ samples of contaminated material showed any growth after 7 days of inoculation indicating that the CNT coated media does possess strong biocidal/biostatic performance properties for relatively long period of time. (See FIG. 11)

TABLE 3
Samples tested for antimicrobial performance

| Sample No | Type of CNT | Type of media | Treatment time/min |
|---|---|---|---|
| 1 | Long | LabX | 1 |
| 2 | Short | DurX | 2 |
| 3 | Short | LabX | 2 |
| 4 | Short | DurX | 12 |
| 5 | Short | LabX | 12 |
| 6 | Short | DurX | 60 |
| 7 | Short | LabX | 60 |

Water Retention

A comparative water absorption test was performed. It was found that the incorporation of carbon nanotubes into the LabX wipe media reduced the water absorption rate. Pristine LabX media absorbed 1 drop of water almost instantly while it took 4-5 seconds for the CNT modified LabX media to absorb a similar drop of water.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the following specification and attached claims are approximations that may vary depending upon the desired properties sought to be obtained by the present invention.

Other embodiments of the invention will be apparent to those skilled in the art from consideration of the specification and practice of the invention disclosed herein. It is intended that the specification and examples be considered as exemplary only, with the true scope of the invention being indicated by the following claims.

What is claimed is:

1. An article comprising carbon nanotubes in an amount sufficient to remove at least one contaminate from a solid surface.

2. The article of claim 1, wherein said carbon nanotubes have at least one functional group, molecule or cluster attached thereto.

3. The article of claim 1, wherein said carbon nanotubes have at least one defect.

4. The article of claim 1, wherein said carbon nanotubes are chosen from single walled, double walled, or multi-walled carbon nanotubes or combinations thereof.

5. The article of claim 1, wherein said carbon nanotubes are at least 5 nm in length.

6. The article of claim 2, wherein said at least one molecule or cluster comprises an organic compound chosen from proteins, carbohydrates, polymers, aromatic or aliphatic alcohols, nucleic acid, or combinations thereof.

7. The article of claim 1, wherein the said contaminants are chosen from fluids, particles, fibers, biological agents, radionuclides, static charge, or combinations thereof.

8. The article of claim 7, wherein said fluids are comprised of water, hydrocarbons, acid, fluids, radioactive wastes, foodstuffs, bases, solvents or combinations thereof.

9. The article of claim 7, wherein the said radionuclides comprise at least one atom or ion chosen from the elements: strontium, iodine, cesium, beryllium, lithium, sodium, barium, polonium, radium, thorium, hydrogen, uranium, plutonium, cobalt, and radon.

10. The article of claim 7, wherein said biological agents comprise molecules chosen from DNA, RNA, and natural organic molecules bacteria, viruses, spores, mold, parasites, pollens, fungi, prion and combinations thereof.

11. The article of claim 10, wherein the bacteria comprises anthrax, coliforms typhus, e-coli, staph, pneumonia, salmonella, or cholera.

12. The article of claim 10, wherein the viruses comprise smallpox, hepatitis, or HIV and their variants.

13. The article of claim 1, wherein said article further comprises a support media for said carbon nanotubes.

14. The article of claim 13, wherein said support media comprises a material chosen from ceramics, carbon or carbon based materials, metals or alloys, polymeric materials, and fibrous materials.

15. The article of claim 14, wherein the fibrous material is paper or a textile comprised of a woven construction, a knit construction, a nonwoven construction, or a combination thereof.

16. The article of claim 14, wherein the textile is comprised of multi-component or bi-component fibers or yarns which may be splittable along their length by chemical or mechanical action.

17. The article of claim 14, wherein the textile is comprised of microdenier fibers.

18. The article of claim 14, wherein the textile is comprised of synthetic fibers, natural fibers, man-made fibers using natural constituent, or blends thereof.

19. The article of claim 18, wherein the synthetic fibers are comprised of polyester, acrylic, polyamide, polyolefin, polyaramid, polyurethane or blends thereof.

20. The article of claim 18, wherein the natural fibers are comprised of wool, cotton, silk, ramie, jute, flax, abaca, wood pulp, or blends thereof.

21. The article of claim 18, wherein the man-made fibers using natural constituents are comprised of regenerated cellulose, lyocell or blends thereof.

22. The article of claim 2, wherein said at least one functional group, molecule or cluster comprises one or more chemical group chosen from hydroxy, hydroxy-alkyl, carboxyls, amines, arenes, nitriles, amides, alkanes, alkenes, alkynes, alcohols, ethers, esters, aldehydes, ketones, polyamides, polyamphiphiles, diazonium salts, metal salts, pyrenyls, thiols, thioethers, sulfhydryls, silanes, and combinations thereof.

23. The article of claim 14, wherein said polymeric materials are chosen from single or multi-component polymers chosen from nylon, acrylic, methacrylic, epoxy, silicone rubbers, polypropylene, polyethylene, polyurethane, polystyrene, aramids, polycarbonates, polychloroprene, polybutylene terephthalate, poly-paraphylene terephtalamide, poly (p-phenylene terephtalamide), and polyester ester ketone, polyesters, polytetrafluoroethylene, polyvinylchloride, polyvinyl acetate, viton fluoroelastomer, polymethyl methacrylate, polyacrylonitrile, and combinations thereof.

24. The article of claim 1, which is in the form of disposable wipe, reusable cloth, article of clothing, swab, mop, brush, pad, or wound dressing.

25. The article of claim 1, wherein said article is anti-microbial, anti-viral, anti-static, or combinations thereof.

26. The article of claim 1, wherein said article is pre-saturated with a liquid to further enhance the removal of a contaminant from a surface.

27. A method of removing at least one contaminant from a solid surface, said method comprising, contacting said solid surface with an article comprising one or more carbon nanotubes.

28. The method of claim 27, wherein said solid surface comprises surfaces, products, equipment, tools, personnel, literature, and biological material in a clean room, industrial environment, clinical environment, household environment, office environment, military environment, public spaces, public transportation, vehicles, and academic environment.

29. The method of claim 27, wherein a liquid is applied to at least one of said article or said solid surface prior to contacting.

30. The method of claim 29, wherein said liquid comprises aqueous or non-aqueous solutions of alcohols, surfactants, detergents, and disinfectants.

31. A method of making an article for capturing and/or removing at least one contaminant from a solid surface, said method comprising:
(a) contacting a support media with a suspension comprising one or more carbon nanotubes to form a carbon nanotube infused support media;
(b) heating said carbon nanotube infused support media to substantially dry said suspension;
(c) rinsing said support to remove loose carbon nanotubes; and
(d) drying said rinsed article.

32. An article for removing at least one contaminant from a solid surface, said article comprising a support media comprising carbon nanotubes in an amount sufficient to remove at least one contaminate from a solid surface, wherein a majority of said carbon nanotubes have at least one defect and/or have at least one functional group, molecule or cluster attached thereto.
